(19) United States
(12) Patent Application Publication
(10) Pub. No.: US 2021/0032211 A1
(43) Pub. Date: Feb. 4, 2021

(54) PROCESSES FOR THE SYNTHESIS OF SULFENTRAZONE

(71) Applicant: FMC Corporation, Phildalephia, PA (US)

(72) Inventors: Wuqiang TU, Shanghai (CN); Bolin FAN, Shanghai (CN); Guanghui LI, Shanghai (CN)

(21) Appl. No.: 16/962,834

(22) PCT Filed: Jan. 18, 2019
(86) PCT No.: PCT/CN2019/072307

§ 371 Date: Jul. 16, 2020

Related U.S. Application Data
(60) Provisional application No. 62/618,692, filed on Jan. 18, 2018.

(51) Int. Cl. C07D 249/12; B01J 31/02

(52) U.S. Cl. CPC C07D 249/12; B01J 31/0271; B01J 31/0244

(57) ABSTRACT

Disclosed are processes for the synthesis of sulfentrazone, which provide a high conversion of sulfentrazone amine and high yield of the final sulfentrazone product.

### FIELD OF THE INVENTION

The present invention relates to the agricultural area, more particularly to a method for the preparation of the herbicide sulfentrazone.

### BACKGROUND OF THE INVENTION

Sulfentrazone is a useful herbicide which was developed and first commercialized by the present applicant. Sulfentrazone is widely used as a safe and efficient herbicide and plays an important role in weed control and crop yield increase.

The synthesis of sulfentrazone is a process that includes the steps of: (a) synthesizing a sulfentrazone amine having the formula as defined below (“formula (i)”) by a known process; and (b) reacting the resultant sulfentrazone amine with methanesulfonyl chloride to obtain the desired sulfentrazone

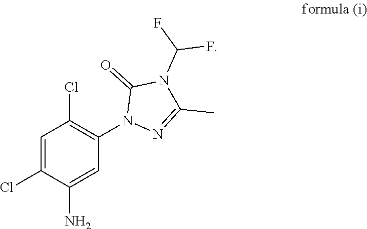 [SMILES: Cc1nn(-c2cc(N)c(Cl)cc2Cl)c(=O)n1C(F)F]

The latter step is a sulfonylation reaction and is normally carried out in the presence of a catalyst. In this regard, many catalysts have been tried and developed and used in the process to improve its yield and efficiency. For example, use of a source of soluble halide (e.g., chloride) and dimethylformamide (DMF) to catalyze the sulfonylation process are taught respectively in U.S. Pat. Nos. 7,169,952 and 5,990,315.

Although some catalysts have been provided and used in the industry, there is still a strong need to provide different and better catalysts to improve the yield and efficiency.

### SUMMARY OF THE INVENTION

Th present invention provides a process for the synthesis of sulfentrazone, which features the use of a new catalyst.

The present invention provides a process for the synthesis of sulfentrazone (“formula (ii)”), comprising reacting a sulfentrazone amine (“formula (i)”) with methanesulfonyl chloride

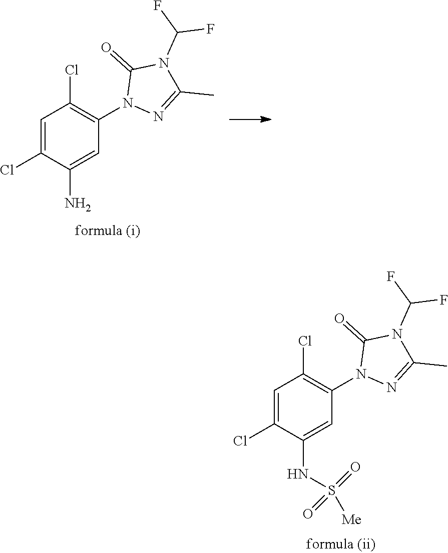 [SMILES: Cc1nn(-c2cc(N)c(Cl)cc2Cl)c(=O)n1C(F)F ; Cc1nn(-c2cc(NS(C)(=O)=O)c(Cl)cc2Cl)c(=O)n1C(F)F]

in the presence of a catalyst selected from imidazole, 1H-1,2,4-triazole, benzimidazole, a compound of Formula-A, a compound of Formula-B or salts thereof

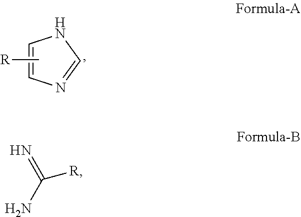

in which

R in both Formulae-A and B each independently represents hydrogen, amino, optionally substituted C₁₋₁₀ alkyl, C₁₋₁₀ haloalkyl, C₁₋₁₀ alkoxy or aryl.

In one aspect, the present invention provides a process for the synthesis of sulfentrazone formula (ii), comprising reacting at elevated temperature the sulfentrazone amine of formula (i) with methanesulfonyl chloride in the presence of imidazole.

### DETAILS OF THE INVENTION

In one aspect, the present invention provides a process for the synthesis of sulfentrazone, comprising reacting at elevated temperature the sulfentrazone amine with methanesulfonyl chloride in the presence of imidazole, 1H-1,2,4-triazole, benzimidazole, a compound having the following Formula-A, or a compound having the following Formula-B or salts thereof

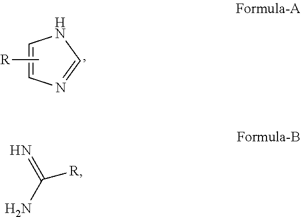

in which

R in both Formulae-A and B each independently represents hydrogen, amino, optionally substituted C₁₋₁₀ alkyl, C₁₋₁₀ haloalkyl, C₁₋₁₀ alkoxy or aryl.

C₁₋₁₀ alkyl can be straight-chain or branched alkyl, such as, methyl, ethyl, n-propyl, i-propyl, butyl, pentyl, hexyl, heptyl, octyl, nonyl, decyl or the different related isomers, e.g. butyl, pentyl or hexyl isomers. In one embodiment, R in Formula-A is methyl or ethyl. In another embodiment, R in Formula-B is methyl or ethyl.

C₁₋₁₀ haloalkyl can be defined as above for alkyl substituted with one or more halo groups, such as fluoro, chloro, bromo or iodo.

C₁₋₁₀ alkoxy can be straight-chain or branched for example, methoxy, ethoxy, n-propyloxy, isopropyloxy and the different butoxy, pentoxy and hexyloxy isomers.

Aryl can be any functional group or substituent derived from an aromatic ring, particulary an aromatic ring structure having 5 to 10 carbon atoms such as phenyl and naphthyl. In one embodiment, R in Formula-A is phenyl. In another embodiment, R in Formula-B is phenyl.

Salts can include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids.

In another aspect, the compound of Formula-A is 2-methylimidazole, 4-methylimidazole, 5-methylimidazole, 2-ethylimidazole, 4-ethylimidazole, 5-ethylimidazole, 2-phenylimidazole, 4-phenylimidazole and 5-phenylimidazole.

In another aspect, the compound of Formula-B is formamidine, acetamidine, or salts thereof. The salts can include hydrochloride, sulfate and phosphate. In another aspect, the compound of Formula-B is formamidine hydrochloride or acetamidine hydrochloride. In another aspect, the compound of Formula-B is formamidine sulfate or acetamidine sulfate, formamidine phosphate or acetamidine phosphate.

For carrying out this process, sulfentrazone amine is first prepared through multiple steps which all are known to those skilled in this art (see U.S. Pat. No. 4,818,275).

In another aspect of the invention, the process is carried out with or without solvent. In one embodiment, the process is carried out in a solvent. The solvent may be selected from aromatic, alkane, and alkene solvents and any mixtures thereof. In one embodiment, the solvent is selected from toluene, xylene, diethylbenzene, and any mixtures thereof. In another embodiment, the solvent is toluene.

The reaction can be carried out over a wide temperature range. One of ordinary skill in the art would recognize that low temperatures can be effective in carrying out the process, but longer reaction times may be encountered; higher temperatures may result in shorter reaction times, but temperatures that are too high may create undesirable results. In one aspect of the invention, the process is carried out at an elevated temperature ranging from about 110° C. to about 160° C. In one embodiment, the temperature ranges from about 120° C. to about 130° C.

In one aspect of the invention, the process is carried out for a period ranging from about 3 to about 12 hours. In one embodiment, the process is carried out for a period ranging from about 4 to about 8 hours. In another embodiment, the reaction is completed in less than 8 hours for the purpose of time efficiency.

In another aspect of the present invention, the process is carried out at atmospheric pressure or at pressures greater than atmospheric pressure. In one embodiment, the process is carried out at greater than atmospheric pressure. In one embodiment, the process is carried out at a pressure ranging from about 0.15 MPa to about 1 MPa. In a further embodiment, the process is carried out at a pressure ranging from 0.15 MPa to 0.5 MPa.

In another aspect, methanesulfonyl chloride and sulfentrazone amine are present in a molar ratio of methanesulfonyl chloride to sulfentrazone amine ranging from about 1 to about 2. In another embodiment, methanesulfonyl chloride is present in molar excess of sulfentrazone amine. In a further preferred embodiment, the molar ratio of methanesulfonyl chloride to sulfentrazone amine ranges from about 1.5 to about 2. In another embodiment, methanesulfonyl chloride is present in excess of the sulfentrazone amine throughout the process. The excess of methanesulfonyl chloride can be maintained throughout the process by adding into the reaction mixture additional methanesulfonyl chloride as needed.

In another aspect, the catalyst such as imidazole is present in at least a catalytic amount. As the term is used herein, “catalytic amount” means an amount that is effective in facilitating the reaction of methanesulfonyl chloride and the sulfentrazone amine In one embodiment, the catalyst is present in an amount ranging from about 0.01 to about 0.2 molar equivalents to sulfentrazone amine In another embodiment, the catalyst is present in an amount ranging from about 0.05 to about 0.15 molar equivalents to sulfentrazone amine. In one embodiment, when imidazole is used as the catalyst, it is present in an amount ranging from about 0.01 to about 0.2 molar equivalents to sulfentrazone amine In another embodiment, the imidazole present ranges from about 0.05 to about 0.15 molar equivalents to sulfentrazone amine In another embodiment, when the catalyst is benzimidazole, 2-methylimidazole, 2-ethylimidazole, 2-phenylimidazole, formamidine hydrochloride or acetamidine

hydrochloride, the catalyst is present in an amount ranging from about 0.01 to about 0.035 molar equivalents to sulfentrazone amine.

The present process can be more cost effective than conventional processes. The catalyst, such as imidazole, is readily available and the present process featuring the use of the new catalyst, such as imidazole, can provide a high conversion of the sulfentrazone amine and a higher yield of the desired sulfentrazone. Without being bound to any theory, the higher yield may be mainly attributed to the avoidance of the formation of the undesired byproduct N-[2,4-dichloro-5-[4-(difluoromethyl)-4,5 -dihydro-3-methyl-5-oxo-1H-1,2,4-triazol-1-yl]phenyl]-formamide, which is a major impurity occurring in the DMF process.

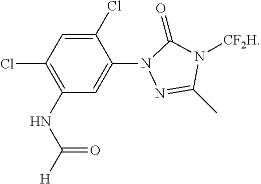 [SMILES: Cc1nn(-c2cc(NC=O)c(Cl)cc2Cl)c(=O)n1C(F)F]

Generally, the present process can provide a high conversion of sulfentrazone amine, such as 99% or higher for imidazole, within a reasonable reaction time, as well as a high yield of sulfentrazone, which can reach up to 90%, 94%, 95%, 96% or even higher.

In another aspect of the present invention, a process is provided for the preparation of a sulfonamide of formula II:

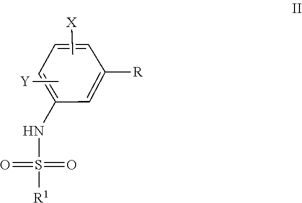

which process comprises reacting at elevated temperature an aniline of formula I:

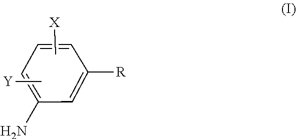

with a suitable sulfonating agent A of the formula R¹—SO₂—Z in the presence of a catalytic amount of a catalyst, such as imidazole;

wherein:
X and Y in both formulae I and II and Z are each independently selected from the group consisting of hydrogen, halo, alkyl, haloalkyl, amino, nitro, alkoxy, hydroxy, anhydridyl, alkylthio, arylthiol, aryloxy, alkylsulfonyl, arylsulfonyl, and substituted or unsubstituted aryl, the substituents comprising one or more members selected from the group consisting of halo,
C₁₋₂₀ alkyl, C₁₋₂₀ alkoxy, nitro, amino, amido, alkylthio, aryl, arylthio, aryloxy, alkylsulfonyl, and arylsulfony;
R in both formulae I and II is selected from the group consisting of hydrogen, alkyl, haloalkyl, aryloxy, substituted or unsubstituted aryl and substituted or unsubstituted heterocyclyl, the substituents comprising one or more members selected from the group consisting of halo, C₁₋₂₀ alkyl, C₁₋₂₀ alkoxy, nitro, amino, amido, alkylthio, aryl, arylthio, aryloxy, alkylsulfonyl, and arylsulfonyl; and
R¹ is selected from the group consisting of hydrogen, alkyl, haloalkyl, and aryl.

Preferred sulfonamides prepared by the present invention are those in which X and Y are halo; R is a substituted or unsubstituted heterocyclyl, the substituents comprising one or more members selected from the group consisting of halo, C₁₋₂₀ alkyl, C₁₋₂₀ alkoxy, nitro, amino, amido, alkylthio, aryloxy, aryl, arylthiol, alkylsulfonyl, and arylsulfonyl; and R¹ is aryl or alkyl.

Particularly preferred sulfonamides prepared by the present invention are those in which X and Y are chloro or fluoro; R is 4-difluoromethyl-4,5-dihydro-3-methyl-5-oxo-1H-1,2,4-triazol-1-yl, 1-methyl-6-trifluoromethyl-2,4-(1H,3H)-pyrimidinedion-3-yl, or 1-amino-6-trifluoromethyl-2,4-(1H,3H)-pyrimidinedion-3-yl; and R¹ is methyl. An even more preferred sulfonamide prepared by the present invention is that in which X is 2-chloro or 2-fluoro, Y is 4-chloro, R is 4-difluoromethyl-4,5-dihydro-3-methyl-5-oxo-1H-1,2,4-triazol-1-yl, and R¹ is methyl.

Preferred anilines that can be used in the present invention are those in which X and Y are halo and R is a substituted or unsubstituted heterocyclyl, the substituents comprising one or more members selected from the group consisting of halo, C₁₋₂₀ alkyl, C₁₋₂₀ alkoxy, nitro, amino, amido, alkylthio, aryloxy, aryl, arylthiol, alkylsulfonyl, and arylsulfonyl.

Particularly preferred anilines that can be used in the present invention are those in which X and Y are chloro or fluoro and R is 4-difluoromethyl-4,5-dihydro-3-methyl-5-oxo-1H-1,2,4-triazol-1-yl, 1-methyl-6-trifluoromethyl-2,4-(1H,3H-pyrimidinedion-3-yl, or 1-amino-6-trifluoromethyl-2,4-(1H,3H)-pyrimidinedion-3-yl. An even more preferred aniline that can be used in the present invention is that in which X is 2-chloro or 2-fluoro, Y is 4-chloro, and R is 4-difluoromethyl-4,5 -dihydro-3 -methyl-5-oxo-1H-1,2,4-triazol-1-yl. Suitable sulfonating agent A that may used in the present invention are those substances that allow for the attachment of a sulfonyl moiety on an amino group. Examples of sulfonating agent A that may be used in the present invention include, but are not limited to, those having the formula R¹—SO₂—Z, wherein R¹ and Z are as defined above. Preferred sulfonating agent A that can be used in the present invention include those agents of the formula R¹—SO₂—Z in which R¹ is aryl or alkyl and Z is halo or anhydridyl. Particularly preferred sulfonating A include those agents of the formula R¹—SO₂-Z in which R¹ is alkyl and Z is halo. An even more preferred sulfonating agent A is an agent of the formula R¹—SO₂-Z in which R¹ is methyl and Z is chloro. “Catalytic amount” as utilized herein shall mean an amount that is effective in facilitating the reaction of aniline and sulfonating agent.

The reaction is preferably carried out at elevated temperature, such as from about 110° C. to about 160° C. more preferably from about 120° C. to about 150° C., preferably for about three to about 12 hours, more preferably for about three to about seven hours. The reaction can be run at lower temperatures, but generally will require an appreciably longer time to complete. In addition, the reaction may be run at atmospheric or increased pressure.

The reaction may be carried out by combining the aniline I with about 1 to about 5, preferably about 1.3 to about 4, molar equivalents of sulfonating agent A to one molar equivalent of aniline I and a catalytic amount, for example, about 0.05 to about 0.15 molar equivalent of catalyst, such as imidazole, to one molar equivalent of aniline I.

In addition, the reaction may be carried out neat or in a solvent. Suitable solvents that can be used in the present invention are those that allow for the formation of a miscible mixture with the aniline of formula I at elevated temperature. Examples of solvents that can be used in the present invention include, but are not limited to, aromatic, alkane or alkene solvents. Preferred solvents that can be used in the present invention are toluene, xylene, and diethylbenzene. A particularly preferred solvent that can be used in the present invention is toluene.

As used in this specification and unless otherwise indicated the substitutent terms “alkyl”, “alkoxy”, “aryloxy”, and “alkoxyarylamino”, used alone or as part of a larger moiety, include straight or branched chains of at least one or two carbon atoms, as appropriate to the substituent, and preferably up to 20 carbon atoms, more preferably up to ten carbon atoms, most preferably up to seven carbon atoms. “Halogen” or “halo” refers to fluorine, bromine, iodine, or chlorine. “Aryl” refers to an aromatic ring structure having 5 to 10 carbon atoms. “Heteroaryl” refers to an aromatic ring structure having 1 to 4 nitrogen, sulfur, or oxygen atoms or a combination thereof as hetero ring components, with the balance being carbon atoms. “High boiling” refers to a compound having a boiling point above 140° C. at ambient pressure. The term “ambient temperature” as utilized herein shall mean a temperature not exceeding 30° C. The term “elevated temperature” as utilized herein shall mean a temperature above ambient temperature, for example, a temperature in the range of about 110° C. to about 160° C.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, process or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The total number of carbon atoms in a substituent group is indicated by the “Cᵢ-Cⱼ” prefix where i and j are numbers from 1 to 10. For example, C₁-C₄ alkyl designates methyl through butyl.

When a compound is substituted with a substituent bearing a subscript that indicates the number of said substituents can exceed 1, said substituents (when they exceed 1) are independently selected from the group of defined substituents. Further, when the subscript indicates a range, e.g. (R)ᵢ₋ⱼ, then the number of substituents may be selected from the integers between i and j inclusive. When one or more positions on a group are said to be “not substituted” or “unsubstituted”, then hydrogen atoms are attached to take up any free valency.

The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

Salts of a compound can include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium.

The present invention is now described in more detail by reference to the following examples, but it should be understood that the invention is not construed as being limited thereto.

### EXAMPLES

### Example 1

### Synthesis of Sulfentrazone Using Sulfentrazone Amine as Starting Material

80.3g of solid sulfentrazone amine (hereinafter “SFTS-NH₂”), and 36.0g of toluene were charged into a 1-liter round-bottomed reaction flask equipped with bottom take-off stopcock. The reaction flask was fitted with a mechanical stirrer, reflux condenser, a thermocouple and a heating mantle Effluent gas was directed to a caustic scrubber containing 10% NaOH. Except as otherwise indicated all reactions of the Examples were carried out at normal atmospheric pressure.

The obtained SFTS-NH₂ in toluene reaction medium was maintained at about 120-130° C., and 40.1 g of methanesulfonyl chloride was slowly charged to the reaction flask. Subsequently, 1.09g of imidazole (equaling 7% molar equivalent of SFTS-NH₂) was added to the medium. The reaction temperature was maintained at about 120-130° C. throughout the reaction.

The reaction was held with agitation under reflux conditions at the temperature of about 120-130° C. until the conversion of SFTS-NH₂ was greater than 99% by GC analysis (i.e., less than 1% unconverted SFTS-NH₂ remains in the reaction medium).

When the reaction was completed, the mixture was cooled slowly to 80° C. and diluted using toluene (400g) to provide a 15 wt % sulfentrazone solution. This diluted mixture was quenched with water, followed by phase separation to collect the organic phase. The organic phase was further subject to crystallization and filtration and the crystals collected were then dried to give 86.5g of solid final product. As analyzed, the weight (percentage) assay was 92%, the isolated solid yield was 89.8%, and the yield in mother liquor (ML) was 5.9%. The overall yield of the final sulfentrazone was calculated to be 95.7%.

### Example 2

### Synthesis of Sulfentrazone Using Imidazole

The procedure of Example 1 to synthesize sulfentrazone was repeated except for varying the imidazole amount and reaction “Time” as set forth in the following Table 1, while keeping the remaining conditions unchanged. The results were summarized as follows. The total yield of sulfentrazone (“SFT”) was calculated as the sum of the “Yield/Solid” and “Yield/ML”. Imidazole residue remaining in the dried solid product was indicated as “Imidazole (ppm)” and the symbol “/” represents Not Measured.

TABLE 1

| | Imidazole | | Imidazole* | Time | Yield/ | Yield/ | Unreacted | Yield/ |
|---|---|---|---|---|---|---|---|---|
| Run | (g) | | (ppm) | (Hrs) | Solid | ML | SFT5-NH2 | SFT |
| 1 | 0.545 | g | 8 | 4 | 88.17% | 3.48% | 0.90% | 91.65% |
| 2 | 0.545 | g | <1 | 4 | 90.16% | 3.25% | 0.89% | 93.41% |
| 3 | 0.545 | g | 21 | 4 | 90.69% | 3.04% | 0.83% | 93.73% |
| 4 | 0.545 | g | / | 9 | 88.94% | 4.61% | 0.78% | 93.55% |
| 5 | 0.545 | g | / | 8 | 89.82% | 3.70% | 0.93% | 93.52% |
| 6 | 0.545 | g | / | 12 | 91.20% | 4.28% | 0.85% | 95.48% |
| 7 | 1.09 | g | 19 | 5 | 90.93% | 3.11% | 0.51% | 94.04% |
| 8 | 1.09 | g | 14 | 6 | 91.34% | 3.28% | 0.45% | 94.62% |
| 9 | 1.09 | g | 10 | 12 | 88.18% | 5.70% | 1.36% | 93.88% |
| 10 | 1.09 | g | 9 | 10 | 90.75% | 5.61% | 0.98% | 96.36% |
| 11 | 1.09 | g | 15 | 8 | 91.80% | 4.53% | 1.03% | 96.33% |
| 12 | 1.09 | g | 7 | 8 | 90.73% | 5.55% | 0.76% | 96.28% |
| 13 | 1.09 | g | 16 | 8 | 89.89% | 5.89% | 0.71% | 95.78% |

*represents the imidazole residual in the obtained sulfentrazone product

### Comparative Example 3

### Synthesis of Sulfentrazone Using Dimethylformamide (“DMF”) as Catalyst

The same procedures of Example 1 were repeated except for replacing imidazole with DMF and varying the reaction “Time” as indicated in the following Table 2. The results were summarized as follows. 0.585g DMF, equaling to 3.5% of SFT-NH₂ by molar equivalence was used in all comparison examples.

TABLE 2

| | | | | | Unreacted | |
|---|---|---|---|---|---|---|
| | DMF | Time/ | Yield/ | Yield/ | SFT-NH₂ | Yield/ |
| Comp. | (g) | (Hrs) | Solid | ML | (wt %) | SFT |
| 1 | 0.585 g | 6 | 85.94% | 5.00% | 1.92% | 90.94% |
| 2 | 0.585 g | 5 | 87.16% | 5.55% | 1.10% | 92.71% |
| 3 | 0.585 g | 4 | 88.47% | 4.65% | 1.57% | 93.12% |
| 4 | 0.585 g | 5 | 87.89% | 3.37% | 1.60% | 91.26% |

As shown in the results of Examples 1 and 2, the present processes using imidazole as catalyst provide a high sulfentrazone amine conversion (99% or even higher), as well as higher sulfentrazone yield (e.g., 94%, 95%, 96% or even higher). In comparison with the DMF process as demonstrated in Example 3, the conversion rate and the sulfentrazone yield are both improved.

These improvements are significant from the perspective of industrial-scale manufacture and bring significant cost reduction. Further, unlike the DMF process where DMF can partially react with sulfentrazone amine, thereby leading to the formation of undesired byproducts and in turn lowering the sulfentrazone yield, imidazole does not react with sulfentrazone amine and therefore unwanted byproducts are avoided.

Another advantage of the present process is that imidazole has a high solubility in water, and thus most of the imidazole is dissolved in the waste water and taken away from the final sulfentrazone product. As shown in Table 1, the residual imidazole in the sulfentrazone solid is less than 25 ppm. That not only simplifies the purification treatment, but also improves the purity of the final sulfentrazone product by reducing byproducts found in the DMF process.

### Example 4

### Synthesis of Sulfentrazone by Varying Catalyst

Repeat the procedure of Example 1 by using different catalysts in replace of imidazole. The following Table 3 outlines the reagents and catalysts used in the present example, and reaction times as well as the conversion rate of from SFT 5-NH₂ to the desired sulfentrazone.

TABLE 3

| | | | | Reaction | Conversion |
|---|---|---|---|---|---|
| | | Amount | | Time | Rate (by |
| | | (g) | Mole | (Hrs) | HPLC) |
| Reagent/ | SFT 5-NH₂ | 31 | 0.10 | / | / |
| Solvent | Toluene | 10 | 0.11 | / | / |
| | Methanesulfonyl | 17.5 | 0.15 | / | / |
| | chloride | | | | |
| Catalyst | Benzimidazole | 0.4 | 0.0035 | 3 | 96.9% |
| | 2-Methylimidazole | 0.3 | 0.0035 | 3 | 98.0% |
| | 2-Ethylimidazole | 0.3 | 0.0035 | 3 | 98.9% |
| | 2-Phenylimidazole | 0.4 | 0.0035 | 3 | 96.8% |
| | Formamidine | 0.3 | 0.0035 | 3 | 92.9% |
| | hydrochloride | | | | |
| | Acetamidine | 0.3 | 0.0035 | 3 | 98.2% |
| | hydrochloride | | | | |

As showed in Table 3, the catalysts of the present invention all provide a high conversion of from SFT 5-NH₂ to the final sulfentrazone product.

What is claimed is:

1. A process for the synthesis of sulfentrazone, comprising reacting sulfentrazone amine of formula (i) with methanesulfonyl chloride 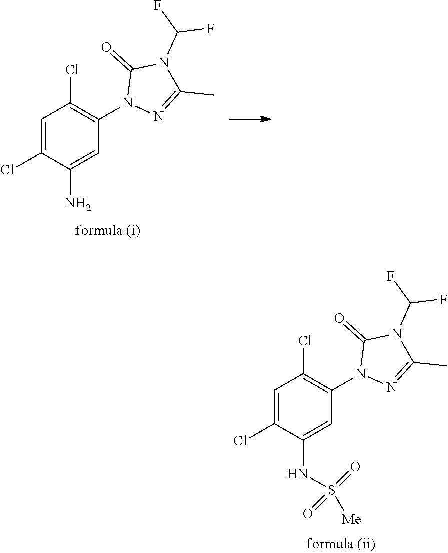 [SMILES: Cc1nn(-c2cc(N)c(Cl)cc2Cl)c(=O)n1C(F)F ; Cc1nn(-c2cc(NS(C)(=O)=O)c(Cl)cc2Cl)c(=O)n1C(F)F]
in the presence of a catalyst selected from imidazole, 1H-1,2,4-triazole, benzimidazole, a compound of Formula-A, a compound of Formula-B or salts thereof 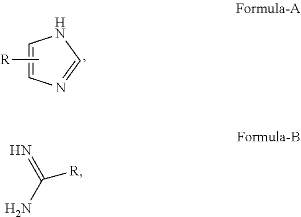
wherein
R in both Formulae-A and B each independently represents hydrogen, amino, optionally substituted C₁₋₁₀ alkyl, C₁₋₁₀ haloalkyl, C₁₋₁₀ alkoxy or aryl.

2. The process of claim 1, wherein the compound of Formula-A is 2-methylimidazole, 4-methylimidazole, 5-methylimidazole, 2-ethylimidazole, 4-ethylimidazole, 5-ethylimidazole, 2-phenylimidazole, 4-phenylimidazole or 5-phenylimidazole.

3. The process of claim 1, wherein the compound of Formula-B is formamidine, acetamidine or salts thereof.

4. The process of claim 3, wherein the compound of Formula-B is formamidine hydrochloride, acetamidine hydrochloride, formamidine sulfate, acetamidine sulfate, formamidine phosphate or acetamidine phosphate.

5. The process of claim 1, wherein the process is carried out in a solvent.

6. The process of claim 2, wherein the solvent is selected from aromatic, alkane, and alkene solvents, and any mixtures thereof.

7. The process of claim 3, wherein the solvent is selected from toluene, xylene, diethylbenzene, and any mixtures thereof.

8. The process of claim 4, wherein the solvent is toluene.

9. The process of claim 1, wherein the process is carried out at an elevated temperature, with the preferred temperature ranging from about 110° C. to about 160° C.

10. The process of claim 6, wherein the temperature ranges from about 120° C. to about 130° C.

11. The process of claim 1, wherein the process is carried out at atmospheric pressure or higher pressure.

12. The process of claim 1, wherein the process is carried out at a pressure ranging from about 0.15 MPa to about 1 MPa.

13. The process of claim 1, wherein the catalyst is present in an amount ranging from about 0.01 to about 0.2 molar equivalents of sulfentrazone amine

14. The process of claim 13, wherein the catalyst is imidazole.

15. The process of claim 13, wherein the catalyst is present in an amount ranging from about 0.05 to 0.15 molar equivalent of sulfentrazone amine

16. The process of claim 15, wherein the catalyst is imidazole.

17. The process of claim 13, wherein the catalyst is benzimidazole, 2-methylimidazole, 2-ethylimidiazole, 2-phenylimidazole, formamidine hydrochloride or acetamidine hydrochloride and the catalyst is present in an amount ranging from about 0.01 to about 0.035 molar equivalents to sulfentrazone amine.

18. The process of claim 1, wherein methanesulfonyl chloride is present in excess of sulfentrazone amine.

19. The process of claim 18, wherein methanesulfonyl chloride is maintained in excess of sulfentrazone amine throughout the process.

20. The process of claim 18, wherein methanesulfonyl chloride and sulfentrazone amine are present in a molar ratio ranging from about 1 to about 2.

21. The process of claim 20, wherein methanesulfonyl chloride and sulfentrazone amine are present in a molar ratio ranging from about 1.5 to about 2.

22. A process for the preparation of a sulfonamide of formula II: 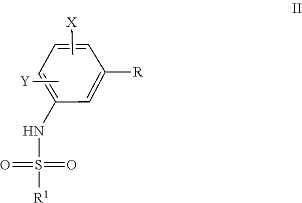
comprising reacting at elevated temperature at least the following: (1) an aniline of formula I: 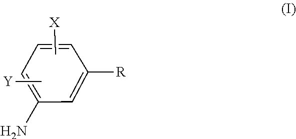
with (2) a sulfonating agent A of the formula R¹—SO₂—Z in the presence of (3) a catalytic amount of a catalyst;
wherein:
the catalyst is imidazole, 1H-1,2,4-triazole, benzimidazole, 2-methylimidazole, 2-ethylimidiazole, 2-phenylimidazole, formamidine hydrochloride or acetamidine hydrochloride;
X and Y in both formulae I and II and Z are each independently selected from the group consisting of hydrogen, halo, alkyl, haloalkyl, amino, nitro, alkoxy, hydroxy, anhydridyl, alkylthio, arylthiol, aryloxy, alkylsulfonyl, arylsulfonyl, and substituted or unsubstituted aryl, the substituents of said substituted aryl comprising one or more members selected from the group consisting of halo, C₁₋₂₀ alkyl, C₁₋₂₀ alkoxy, nitro, amino, amido, alkylthio, aryl, arylthio, aryloxy, alkylsulfonyl, and arylsulfony;
R in both formulae I and II is selected from the group consisting of hydrogen, alkyl, haloalkyl, aryloxy, substituted or unsubstituted aryl and substituted or unsubstituted heterocyclyl, the substituents of said substituted aryl or heterocyclyl comprising one or more members selected from the group consisting of halo, C₁₋₂₀ alkyl, C₁₋₂₀ alkoxy, nitro, amino, amido, alkylthio, aryl, arylthio, aryloxy, alkylsulfonyl, and arylsulfonyl; and
R¹ is selected from the group consisting of hydrogen, alkyl, haloalkyl, and aryl.

23. The process of claim 22, wherein the catalyst is imidazole.
